(19) United States
(12) Patent Application Publication
(10) Pub. No.: US 2024/0315630 A1
(43) Pub. Date: Sep. 26, 2024

(54) VISUALIZING LOCAL QUALITY ON AN ELECTROPHYSIOLOGICAL MAP

(71) Applicant: BIOSENSE WEBSTER (ISRAEL) LTD., Yokneam (IL)

(72) Inventors: Jonathan Yarnitsky, Haifa (IL); Shaked Weiss, Kiryat Ata (IL); Meytal Segev, Haifa (IL)

(21) Appl. No.: 18/393,772

(22) Filed: Dec. 22, 2023

Related U.S. Application Data
(60) Provisional application No. 63/448,416, filed on Feb. 27, 2023.

(51) Int. Cl. A61B 5/287; A61B 5/339

(52) U.S. Cl. CPC A61B 5/287; A61B 5/339

(57) ABSTRACT

Methods and systems indicate where in the EP map the quality of the EP values is below a required level. Indications provide real-time visual aid to the physician, helping him or her to assess in which areas of the cardiac chamber additional EP signals need to be acquired in order to achieve the desired quality and sufficient spatial coverage.

### CROSS-REFERENCE TO RELATED APPLICATIONS

This application claims the benefit of U.S. Provisional Patent Application 63/448,416, filed Feb. 27, 2023, whose disclosure is incorporated herein by reference.

### FIELD OF THE DISCLOSURE

The present disclosure relates generally to electrophysiological mapping, and particularly to visualization of cardiac electrophysiological maps.

### BACKGROUND

Electrophysiological (EP) signals acquired from a patient are commonly visualized on a display device. For example, U.S. Pat. No. 11,478,182, whose disclosure is incorporated herein by reference, describes a method that includes receiving (i) a modeled surface of at least a portion of a heart and (ii) multiple EP values measured at multiple respective positions in the heart. Multiple regions are defined on the modeled surface and, for each region, a confidence level is estimated for the EP values whose positions fall in the region. The modeled surface is presented to a user, including (i) the EP values overlaid on the modeled surface, and (ii) the confidence level graphically visualized in each region of the modeled surface.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

The present disclosure will be more fully understood from the following detailed description of the examples thereof, taken together with the drawings in which:

### BRIEF DESCRIPTION OF THE DRAWINGS

FIG. 1 is a schematic pictorial illustration of a catheter-based electrophysiology mapping and ablation system, in accordance with an example of the disclosure;

FIG. 2 is a schematic representation of a display showing an electroanatomical map of a cardiac chamber, in accordance with an example of the disclosure; and

FIG. 3 is a flow chart that schematically illustrates a process for displaying quality metrics on an electroanatomical map, in accordance with an example of the present disclosure.

### DETAILED DESCRIPTION OF EXAMPLES

### Overview

Cardiac diagnostic and therapeutic systems may acquire multiple intracardiac electrophysiological (EP) signals during an invasive procedure. Such systems acquire the EP signals using multiple electrodes fitted at the distal end of a probe. The acquired EP signals may be analyzed to compute EP values such as, for example, local activation times (LATs), which enable a physician to assess the condition of the tissue of the cardiac chamber. An EP map of the cardiac chamber may be displayed on a display device, with the computed EP values indicated on the map by different colors overlaid on the anatomy of the chamber. (This sort of map is also referred to as an electroanatomical map.)

However, due to factors such as the stability and sharpness of the signals, stability of the catheter position, effects of respiration, and the proximity of the probe to the cardiac tissue, the quality and density of the extracted EP values may vary. Consequently, the physician may not be aware of the varying quality of the displayed EP values solely based on the color map of these values.

Examples of the present disclosure that are described hereinafter provide methods and systems to indicate rapidly and clearly where in the EP map the quality of the EP values is below a required level. This kind of indication provides a real-time visual aid to the physician, helping him or her to assess in which areas of the cardiac chamber additional EP signals need to be acquired in order to achieve the desired quality and sufficient spatial coverage.

In an example of the present disclosure, a system receives multiple EP data points with their respective locations and EP values, generated from signals acquired by one or more electrodes of a catheter in contact with tissue of a cardiac chamber. The system scores each of the received data points with a quality score, divides the map of the cardiac chamber to regions, and computes a quality metric for each region based on the quality scores of the data points in the region. The system displays an EP map, which is colored with hues representing the EP values, while modulating the color of each region according to the quality metric of the region.

In one example the system modulates the luminance (brightness) of each region so that a lower quality is indicated by a darker region. In another example, the system modulates the saturation of the hue of a region so that a lower saturation (“bleached-out” hues) indicate a lower quality. Modulation of this sort gives the physician clear feedback in real time as to the quality of EP data on the map and helps him/her decide rapidly in which regions of the cardiac chamber additional EP data should be collected.

### System Description

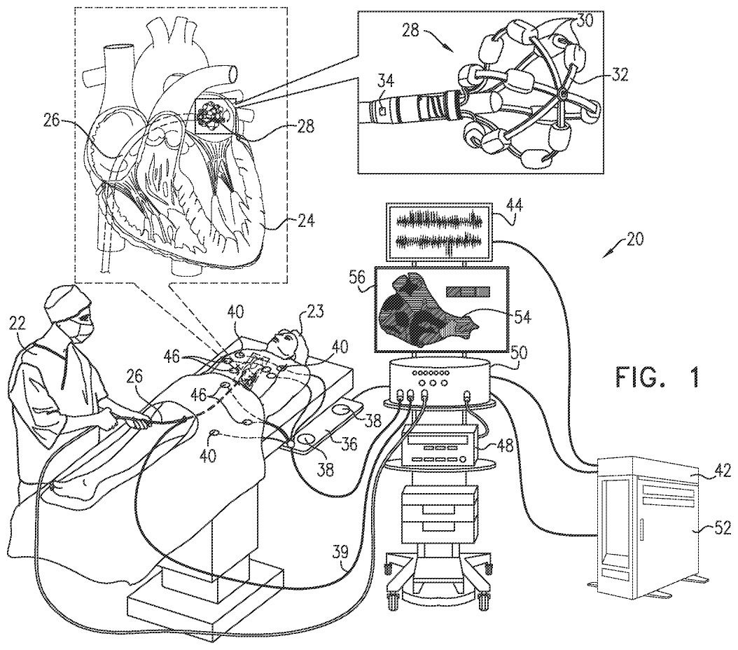

FIG. 1 shows an example catheter-based electrophysiology mapping and ablation system 20, in accordance with an example of the disclosure. System 20 may include multiple catheters, which are percutaneously inserted by a physician 22 through the vascular system of a patient 23 into a chamber or vascular structure of a heart 24. Typically, a delivery sheath (not shown) is inserted into the left or right atrium near a desired location in heart 24. Thereafter, one or more catheters 26 are inserted through the delivery sheath so as to arrive at the desired location in heart 24. The multiple catheters may include catheters dedicated for sensing intracardiac electrogram (IEGM) signals, catheters dedicated for ablating, and/or catheters used for both sensing and ablating. The distal part of catheter 26 in the pictured example comprises a basket assembly 28. Physician 22 may manipulate catheter 26 to place basket assembly 28 in contact with the heart wall for sensing a target site in heart 24 and/or for ablating tissue at the target site.

Catheter 26 is an exemplary catheter that includes multiple electrodes 30 distributed over a plurality of spines 32 in basket assembly 28 and configured to sense IEGM signals and/or ablate myocardial tissue. Catheter 26 additionally includes one or more position sensors 34 embedded in the distal part of the catheter for tracking the position and orientation of basket assembly 28, as described further hereinbelow. For example, position sensor 34 may comprise a magnetic position sensor including three magnetic coils for sensing three-dimensional (3D) position and orientation.

Magnetic position sensor 34 may be operated together with a location pad 36 including multiple magnetic coils 38 configured to generate magnetic fields in a predefined working volume containing heart 24. The position of basket assembly 28 of catheter 26 may be tracked based on magnetic fields generated by location pad 36 and sensed by magnetic position sensor 34 (which may include three orthogonal coils). Details of magnetic position sensing technology that may be applied for this purpose are described, for example, in U.S. Pat. Nos. 5,5391,199; 5,443,489; 5,558,091; 6,172,499; 6,239,724; 6,332,089; 6,484,118; 6,618,612; 6,690,963; 6,788,967; and 6,892,091.

System 20 optionally includes one or more electrode patches 40 in contact with patient 23 to establish location references for location pad 36, as well as for impedance-based tracking of electrodes 30. For impedance-based tracking, electrical current is directed to electrodes 30 and sensed at electrode patches 40 so that the location of each electrode 30 can be triangulated via electrode patches 40. Details of this sort of impedance-based location tracking technology are described in U.S. Pat. Nos. 7,536,218; 7,756,576; 7,848, 787; 7,869,865; and 8,456,182.

A recorder 42 records EP signals such as electrograms 44 captured by body-surface ECG electrodes 46 and intracardiac electrograms (IEGM) captured by electrodes 30 of catheter 26. Recorder 42 may include pacing capability for pacing the heart rhythm and/or may be electrically connected to a standalone pacer.

System 20 may include an ablation energy generator 48 for providing ablative energy to one or more of electrodes 30. Energy produced by ablation energy generator 48 may include, but is not limited to, radiofrequency (RF) energy or pulsed-field ablation (PFA) energy, including monopolar or bipolar high-voltage DC pulses as may be used to effect irreversible electroporation (IRE), or combinations thereof.

A patient interface unit (PIU) 50 comprises an interface for electrical communication between catheters 26, other electrophysiological equipment, a power supply, and a workstation 52 for controlling operation of system 20. Electrophysiological equipment in system 20 may include for example, multiple catheters 26, location pad 36, body surface ECG electrodes 46, electrode patches 40, ablation energy generator 48, and recorder 42. Optionally, PIU 50 additionally includes processing capability for implementing real-time computations of the position of the catheters and for processing ECG signals.

Workstation 52 includes a memory and a processor, with appropriate operating software stored in the memory, and user interface capability. Workstation 52 may provide multiple functions, optionally including (1) modeling the endocardial anatomy in three-dimensions (3D) and rendering the model or an anatomical map 54 for display on a display device 56; (2) displaying on display device 56 EP data, such as LATs, unipolar potentials, or bipolar potentials compiled from EP signals such as recorded electrograms 44, using representative colors or other visual indicia or imagery superimposed on the rendered anatomical map 54; (3) displaying real-time location and orientation of one or more catheters within heart 24; and (4) displaying on display device 56 sites of interest such as places where ablation energy has been applied. A commercial product embodying elements of system 20 is the CARTO® 3 System, available from Biosense Webster, Inc. (31A Technology Drive, Irvine, CA 92618).

### Method of Scoring and Displaying Ep Data Quality

Workstation 52 computes for each EP data point a quality score (QS), indicating a level of confidence that the computed value of the data point accurately represents the true EP value in the tissue. Any suitable scoring scheme may be used for this purpose. In the present example, the QS is composed of a “smart index” (SI) for each data point and of a local density (DENS) of data points. SI is formed by a weight function given by Eq. 1 below, wherein the weight function weighs several quality parameters of an EP data point, as listed, for example, in Table I below. Each data point receives a value of SI ranging between 0 and 1.

The SI of an EP data point is given by:

$$SI = \frac{{\sum }_{n = 1}^{N} {par ameter\_weight}_{n} * {par ameter\_score}_{n}}{{\sum }_{n = 1}^{N} {par ameter\_weight}_{n}} , \qquad Eq . 1$$

Here N is the total number of parameters (see Table I of parameters below), with each parameter given a weight, e.g., a score from 1 to 5. Again, each parameter's normalized score is a number ranging between 0 and 1.

TABLE I
SCORING PARAMETERS

| Parameter | Parameter | |
|---|---|---|
| Name | Weight | Parameter Score |
| Pattern | 5 | (−1) to 1 |
| matching | | According to the PM correlation |
| | | score |
| CL | 2 | $\vert Current CL - medium CL \vert ≡ Δ$ ${σ}^{2} = CL variance$ $score = \{ \begin{matrix}1 - \frac{Δ}{{σ}^{2}} , & current CL is in range \\ 0 , & current CL is out range\end{matrix}$ |
| LAT stability | 3 | $\vert Current LAT - Prev . beat LAT \vert ≡ Δ \\ score = \{ \begin{matrix}1 - \frac{Δ}{12} , & Δ \leq 12 \\ 0 , & Δ > 12\end{matrix}$ |
| Complex | 5 | 1/0 |
| data point | | |
| (e.g., LAM, | | |
| CFAE) | | |
| TPI | 4 | 1 (touch)/0 (no touch or unknown) |
| Position stability | 3 | $\vert Current position - Prev . beat position \vert ≡ Δ$ $score = \{ \begin{matrix}1 - \frac{Δ}{10} , & Δ \leq 10 \\ 0 , & Δ > 10\end{matrix}$ |
| Respiration | 5 | 1/0 (in/out respiration threshold) |
| gating | | |
| Location | 2 | 0.2 |
| (CPM) | | |
| SNR | 2 | $score = \{ \begin{matrix}\begin{matrix}\frac{S N R}{10} , & SNR \leq 10\end{matrix} \\ 1 , S N R > 10\end{matrix}$ |
| $\vert \frac{d V}{d t} \vert$ | 2 | $score = \{ \begin{matrix}\begin{matrix}\vert \frac{d V}{d t} \vert / 5 , & \vert \frac{d V}{d t} \vert \leq 5\end{matrix} \\ 1 , \vert \frac{d V}{d t} \vert > 5\end{matrix}$ |

In Table I, complex fractionated atrial electrograms (CFAE) and late activation mapping (LAM) indicate complex EP signal behavior, such as those from a scarred tissue region. Because this deserves focus from the physician, this distinction may therefore receive a high weight, at least for some arrhythmias.

In Table I, pattern matching is a test of data point consistency over cardiac cycles (a value of correlation between cardia cycles).

CL stands for cardiac cycle length.

TPI stands for touch pressure index (or contact pressure index), which estimates contact force of a catheter electrode with a tissue wall during acquisition of the data point.

Position stability is a measure of electrode position consistency during acquisition over two or more cardiac cycles.

Respiratory weights adversely impact respiration on acquisition, such as introducing noise to signals.

Location CPM is a measure of the aforementioned ACL position tracking technique on electrode location consistency during acquisition.

SNR stands for the signal to noise ratio, which, if low, indicates a less robust acquisition.

$$\vert \frac{d V}{d t} \vert$$

reflects the sharpness of the signal deflections.

Table I is brought by way of example only. Other parameter lists may be different.

A local density (DENS) for a given data point is defined as the number of data points acquired within a specified radius (R) of the location of the given data point. A relative density (DENS_{REL}) is defined as DENS_{REL}=DENS/DENS_{REQ}, where DENS_{REQ} is a required local density for acceptable sampling. Both the radius R and the required local density DENS_{REQ} are determined either automatically by workstation 52 or by physician 22. For EP data points, where DENS_{REL} would compute to a value higher than 1, a value of DENS_{REL}=1 is assigned. Alternatively, other definitions for local density DENS may be used.

For each EP point, workstation 52 computes a quality score QS as a weighted average from SI and from DENS_{REL}, as given by Eq. 2:

$$QS = \frac{{w}_{SI} S I + {w}_{DENS} {DENS}_{REL}}{{w}_{SI} + {w}_{DENS}} \qquad Eq . 2$$

The weights W_{SI} and W_{DENS} are determined either automatically by workstation 52 or by physician 22.

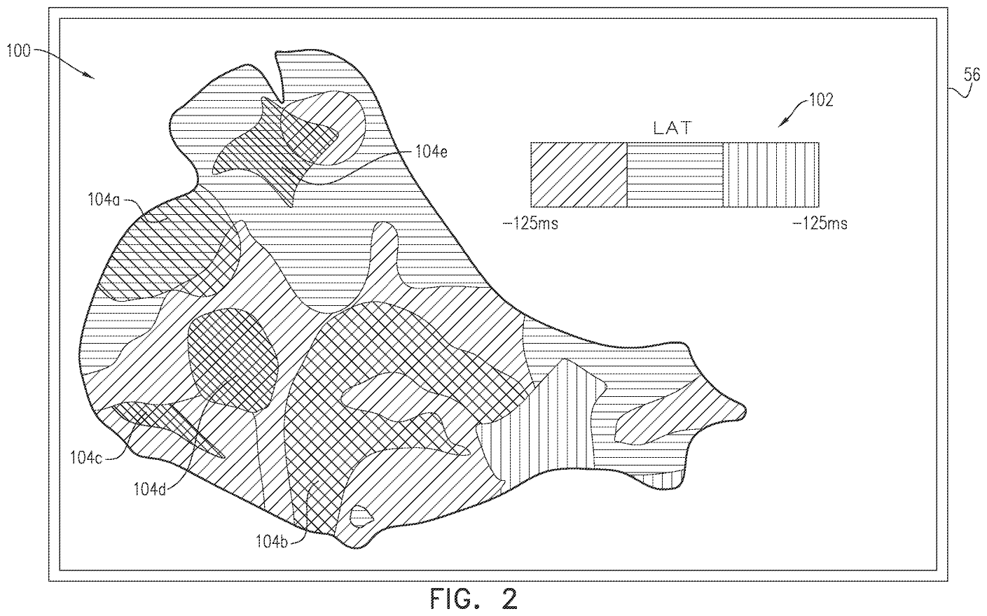

FIG. 2 is a schematic representation of display device 56 showing LAT map 100 of a cardiac chamber, with regions of the map modulated according to a quality metric of a respective region, in accordance with an example of the disclosure. Alternatively, other EP values, for example bipolar or unipolar potentials, could be displayed in similar fashion.

The LAT values of map 100 are indicated by shading according to a key 102, wherein the different shadings show LAT-values ranging from −125 ms to +125 ms. For the sake of clarity of the figure, map 100 is a simplified rendition of an LAT map. In a commonly used color display, the LAT-values are mapped to a continuous or semi-continuous map of hues, thus displaying a far larger number of levels of LAT than the three levels of map 100. Furthermore, the actual color map does not generally have sharp borders between different hues as illustrated in FIG. 2. Rather, the hues are interpolated and blended over the surface of the map to represent the gradual variation of the LAT over the surface of the cardiac chamber.

Workstation 52 has computed quality metrics for the EP data shown in map 100, for example as an average of the quality scores of the EP data points within the region. On this basis, workstation 52 has identified and marked five regions 104a-104e having low quality metrics. The division into regions and their effect on the luminance of the region is described hereinbelow:
Any area in map 100, wherein the quality metric is above 0.8, is excluded from regions 104a-104e. In these areas, the luminance is at its full value on display device 56;
In regions 104a and 104b, the quality metric is equal to or below 0.8, but above 0.5. In these regions, the respective luminance values are reduced to 60% of their full values, indicated in map 100 by a medium density of the cross-hatching lines; and
In regions 104c-104e the quality metric is equal to or below 0.5. In these regions, the luminance values are reduced to 30% of their full values, indicated by a heavy density of the cross-hatching lines.

For the sake of simplicity, only three levels of the quality metric are indicated in map 100. For a conventional color display with a high dynamic range of luminance, a higher number of levels may be used.

Alternatively to the values used in the algorithm described hereinabove, other limits for the quality metric and other reductions of luminance may be applied. Furthermore alternatively, rather than modulating the luminance, the saturation of the hues of map 100 may be modulated responsively to the quality metrics of the regions, or both the luminance and the saturation may be modulated. Further additionally or alternatively, the transparency of map 100 may be modified as a function of the quality metrics, so that regions are rendered as more transparent or more opaque depending on the quality.

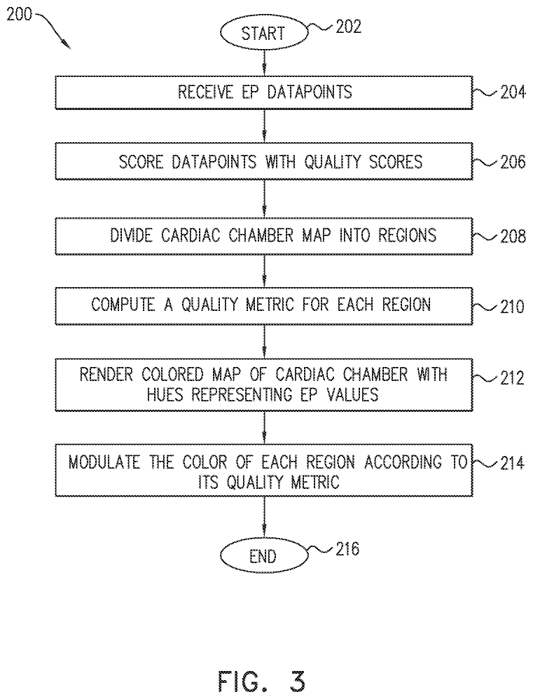

FIG. 3 is a flow chart 200 that schematically illustrates a process for displaying quality metrics on an LAT map of a cardiac chamber, in accordance with an example of the present disclosure. For the sake of concreteness and clarity, the description below assumes that the process is carried out by workstation 52, under the control of appropriate software program instructions. The software is typically stored on tangible, non-transitory computer-readable media, such as optical, magnetic, or electronic memory media. Alternatively, the method may be carried out by other processors, in different system configurations.

The process starts at a start step 202. In a data receiving step 204, workstation 52 receives multiple EP datapoints, wherein each data point comprises an EP value and a location of the data point in the cardiac chamber. In a scoring step 206, workstation 52 computes a quality score for each EP datapoint, for example using the scoring scheme described above or any other suitable algorithm. In a region division step 208, workstation 52 divides the map of the cardiac chamber into regions. In a quality metric step 210, workstation 52 computes a quality metric for that region based on the quality scores of the EP datapoints within each region.

In a map rendering step 212, workstation 52 renders a colored anatomical map of the cardiac chamber on display device 56, wherein the hues of the map represent the EP values in each location of the map. In a color modulation map 214, workstation 52 modulates the color of each region of the map according to its quality metric, thus displaying to physician 22 a map of the EP values with a clear visual indication of the quality of the EP datapoints within the map. For this purpose, for example, workstation 52 overlays a graphical layer on the color map having a property that varies as a function of the quality metrics. This overlay may reduce the luminance and/or the saturation of the color as a function of decreasing quality in regions of the map.

The process ends in an end step 216. As the physician acquires additional data points (particularly in regions of low quality), workstation 52 may modify the colors in the map to reflect the newly measured values and the resulting improved measurement quality. The physician may continue this process until the entire map has been colored with high quality.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. A method for generating an electrophysiological (EP) map, comprising:
receiving multiple EP data points comprising respective locations and EP values, generated from signals acquired by one or more electrodes of a catheter that are in contact with tissue of a cardiac chamber;
scoring the received data points with respective quality scores;
computing respective quality metrics for multiple regions of the cardiac chamber responsively to the quality scores of the data points in the regions; and
rendering a map of the cardiac chamber to a display while coloring the regions of the map with hues selected responsively to the EP values of the data points in the regions and modulating respective colors of the regions responsively to the respective quality metrics.

2. The method according to claim 1, wherein modulating the respective colors of the regions comprises reducing a luminance of a color as a function of decreasing quality.

3. The method according to claim 1, wherein modulating the respective colors of the regions comprises reducing a saturation of a color as a function of decreasing quality.

4. The method according to claim 1, wherein modulating the respective colors of the regions comprises overlaying on the map a graphical layer having a property that varies as a function of the quality metrics.

5. The method according to claim 1, wherein rendering the map comprises modifying a transparency of the regions as a function of the quality metrics.

6. The method according to claim 1, wherein the EP values comprise local activation times (LATs).

7. The method according to claim 1, wherein the EP values comprise bipolar potentials.

8. The method according to claim 1, wherein the EP values comprise unipolar potentials.

9. The method according to claim 1, wherein computing the quality metrics comprises averaging the quality scores of the data points within each of the regions.

10. The method according to claim 1, wherein scoring the received data points comprises computing a density of the data points in each of the locations.

11. Medical apparatus, comprising:
a display; and
a processor configured to:
receive multiple e EP data points comprising respective locations and EP values, generated from signals acquired by one or more electrodes of a catheter that are in contact with tissue of a cardiac chamber;
score the received data points with respective quality scores;
compute respective quality metrics for multiple regions of the cardiac chamber responsively to the quality scores of the data points in the regions; and
render a map of the cardiac chamber to the display while coloring the regions of the map with hues selected responsively to the EP values of the data points in the regions and modulating respective colors of the regions responsively to the respective quality metrics.

12. The apparatus according to claim 11, wherein modulating the respective colors of the regions comprises reducing a luminance of a color as a function of decreasing quality.

13. The apparatus according to claim 11, wherein modulating the respective colors of the regions comprises reducing a saturation of a color as a function of decreasing quality.

14. The apparatus according to claim 11, wherein modulating the respective colors of the regions comprises overlaying on the map a graphical layer having a property that varies as a function of the quality metrics.

15. The apparatus according to claim 11, wherein the processor is configured to modify a transparency of the regions in the rendered map as a function of the quality metrics.

16. The apparatus according to claim 11, wherein the EP values comprise local activation times (LATs).

17. The apparatus according to claim 11, wherein the EP values comprise bipolar potentials.

18. The apparatus according to claim 11, wherein the EP values comprise unipolar potentials.

19. The apparatus according to claim 11, wherein computing the quality metrics comprises averaging the quality scores of the data points within each of the regions.

20. The apparatus according to claim 11, wherein scoring the received data points comprises computing a density of the data points in each of the locations.

21. A computer software product comprising a non-transitory computer-readable medium in which program instructions are stored, which instructions, when read by a computer, cause the computer receive multiple EP data points comprising respective locations and EP values, generated from signals acquired by one or more electrodes of a catheter that are in contact with tissue of a cardiac chamber, to score the received data points with respective quality scores, to compute respective quality metrics for multiple regions of the cardiac chamber responsively to the quality scores of the data points in the regions, and to render a map of the cardiac chamber to a display while coloring the regions of the map with hues selected responsively to the EP values of the data points in the regions and modulating respective colors of the regions responsively to the respective quality metrics.
